(19) United States
(12) Patent Application Publication
(10) Pub. No.: US 2024/0177854 A1
(43) Pub. Date: May 30, 2024

(54) MONITORING REMOTE CONTROL SUITABILITY

(71) Applicant: Siemens Healthcare GmbH, Erlangen (DE)

(72) Inventors: Elisabeth Preuhs, Erlangen (DE); Christian Kaethner, Freiburg (DE); Marcus Pfister, Bubenreuth (DE); Markus Kowarschik, Erlangen (DE); Anne Tjorven Büßen, Erlangen (DE)

(21) Appl. No.: 18/523,864

(22) Filed: Nov. 29, 2023

(30) Foreign Application Priority Data
Nov. 30, 2022 (DE) 10 2022 212 852.4

(51) Int. Cl. G16H 40/67; G06Q 10/06; G16H 10/60; G16H 20/00; G16H 40/20

(52) U.S. Cl. CPC G16H 40/67; G06Q 10/06; G16H 10/60; G16H 20/00; G16H 40/20

(57) ABSTRACT

A method for monitoring remote control suitability includes capturing an instantaneous suitability parameter of a member of remote operating staff. The member of remote operating staff is equipped to remotely control a medical device by a remote control unit. The remote control unit is configured to provide a control signal to the medical device. The suitability parameter is provided by the remote control unit and/or a sensor unit for capturing the member of remote operating staff. The method includes identifying, based on the suitability parameter, whether a reduction in the suitability of the member of remote operating staff exists. In the case of affirmation, the medical device is provided with an alternative control signal, and in the case of negation, the medical device is provided with the control signal.

This application claims the benefit of German Patent Application No. 10 2022 212 852.4, filed on Nov. 30, 2022, which is hereby incorporated by reference in its entirety.

### BACKGROUND

The present embodiments relate to a method for monitoring remote control suitability, to a system, and to computer program product.

With minimally invasive interventions, therapies (e.g., placing of stents) or diagnoses (e.g., detection of stenoses) are frequently carried out by medical objects introduced into the body. These medical objects are customarily advanced via guide wires and catheters up to their site of operation through a point of access in the groin (e.g., the Arteria femoralis) or the left armpit (e.g., radial points of access via the Arteria suclavia). The navigation into the individual vessel branches frequently takes place by twisting and advancing the guide wire or catheter at the entry point.

If such procedures are now supported by a robotic movement apparatus (e.g., a catheter robot and/or a vascular navigation robot), the movement apparatus, remotely controlled by a member of medical operating staff (e.g., a female or male doctor) frequently takes over the manipulation of the medical object.

Depending on the application, the medical operating staff may be situated in the same room as the movement apparatus or in an appropriate room in a hospital that is far away. In the second case, two people or groups in “distributed” teams frequently have to work together, a member of on-site operating staff in situ at the examination object and a member of remote operating staff for remote control of the movement apparatus, which is arranged at a distance from the examination object. The member of on-site and/or remote operating staff may also include just one person here.

It is possible to use different tools to enable or support communication between the distributed teams during a remote-controlled procedure (e.g., remote procedure). For example, bidirectional real-time image transfers, additional items of information (e.g., about medical instruments currently being used or the positioning thereof), as well as vital parameters of the examination object may be provided. A reduction in the suitability of the member of remote operating staff (e.g., a medical emergency and/or a technical failure) may result in incorrect operation of the movement apparatus. The examination object may be disadvantageously injured hereby.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, suitability of a member of remote operating staff during a remote procedure is monitored.

In a first aspect, the present embodiments relate to a method for monitoring remote control suitability. In a first act a), an instantaneous suitability parameter of a medical member of remote operating staff is captured. The medical member of remote operating staff is equipped to remotely control a medical device using a remote control unit. The remote control unit is configured to provide a control signal to the medical device. In addition, the suitability parameter is provided by the remote control unit and/or a sensor unit for capturing the member of remote operating staff. In a second act b), it is identified based on the suitability parameter whether a reduction in the suitability of the member of remote operating staff exists. In the case of affirmation, the medical device is provided with an alternative control signal. In the case of negation, the medical device is provided with the control signal.

The member of remote operating staff may include one or more persons (e.g., a female or male doctor). Further, the member of remote operating staff may be equipped to remotely control the medical device using the remote control unit.

The remote control unit may include a user interface (e.g., an input unit and an output unit, such as a display unit). The input unit may be configured to capture a user input of the member of remote operating staff. The input unit may include, for example, a keyboard and/or a button and/or a joystick and/or a touchpad and/or a microphone (e.g., for speech recognition) and/or a camera (e.g., for capturing gestures). In one embodiment, the input unit may be integrated in the display unit (e.g., in the case of a resistive and/or capacitive input display (touchscreen)). The remote control unit may be arranged to be spatially and/or locally spaced apart (e.g., distant) from the medical device (e.g., in a different room or building). The remote control unit may provide the medical device with the control signal as a function of the user input. The medical device may be configured to receive the control signal from the remote control unit. Further, the medical device may be remotely controlled by the remote control unit (e.g., by the control signal).

The control signal may have instructions (e.g., control commands) and/or a sequence of instructions that prompt the medical device to execute a procedural step on an examination object (e.g., a movement and/or positioning of the medical device and/or recording of image data and/or triggering of a treatment). The medical device may include, for example, a medical imaging device and/or a medical treatment device (e.g., a surgical robot and/or catheter robot and/or a brachytherapy device). The examination object may be, for example, a female human and/or animal patient and/or a male human and/or animal patient and/or an examination phantom.

The suitability parameter may characterize a, for example, procedure-specific, instantaneous suitability of the member of remote operating staff for monitoring and/or remotely controlling the medical device. The suitability parameter may have a value (e.g., a Boolean value and/or a numerical value and/or a letter classification) that rates the instantaneous suitability of the member of remote operating staff (e.g., in a procedure-specific manner; on a specified rating scale) in a specified rating standard or binarily. The suitability of the member of remote operating staff may include a bodily (e.g., physical) and/or cognitive and/or technical suitability. A bodily restriction (e.g., a motor restriction) and/or a cognitive restriction (e.g., a loss of consciousness) and/or a lack of expertise of the member of remote operating staff may result in a reduction in suitability (e.g., in a lower rating of the suitability of the member of remote operating staff by the suitability parameter).

In one embodiment, the instantaneous suitability parameter may be provided by the remote control unit. For example, the remote control unit may provide the instantaneous suitability parameter based on the user input (e.g., an interval between successive user inputs and/or a type of user input and/or an absence of the user input).

Alternatively or in addition, the instantaneous suitability parameter may be provided by the sensor unit for capturing the member of remote operating staff. The sensor unit may include, for example, an optical sensor (e.g., a camera) and/or an acoustic sensor (e.g., a microphone) and/or a mechanical sensor (e.g., a push button) that is configured to capture the member of remote operating staff (e.g., a positioning and/or change in positioning and/or a bodily and/or cognitive state of the member of remote operating staff). In one embodiment, the sensor unit may be at least partially (e.g., fully) integrated in the remote control unit or be arranged spaced apart from the remote control unit. In addition, the sensor unit may provide the instantaneous suitability parameter as a function of the captured positioning and/or change in positioning and/or of the captured bodily and/or cognitive state of the member of remote operating staff.

In act b), it is possible to identify, based on the suitability parameter, whether a reduction in suitability (e.g., a loss of suitability) of the member of remote operating staff exists. Act b) may include a comparison of the instantaneous suitability parameter with a previously captured (e.g., historic) suitability parameter of the member of remote operating staff and/or a specified desired suitability parameter (e.g., a minimum suitability parameter). The comparison may include, for example, a formation of a difference or quotient. Undershooting of the minimum suitability parameter may be identified, for example, as the existence of a reduction in suitability. Alternatively or in addition, a reduction in the instantaneous suitability parameter with respect to the previously captured (e.g., historic) suitability parameter of the member of remote operating staff may be identified as a reduction in suitability.

In case of affirmation (e.g., with the existence of a reduction in the suitability of the member of remote operating staff), the medical device may be provided with an alternative control signal. The alternative control signal may have, for example, all features and properties of the control signal. The alternative control signal may be configured for remotely controlling the medical device. Alternatively or in addition, the alternative control signal may have an item of information on the reduction in the suitability of the member of remote operating staff.

In case of negation (e.g., with the non-existence of a reduction in the suitability of the member of remote operating staff), the medical device may be provided with the control signal.

Providing the alternative control signal and/or the control signal may include, for example, storage on a computer-readable storage medium and/or transfer to a provisioning unit and/or the medical device.

The embodiment makes it possible to monitor the suitability of the member of remote operating staff during a remote procedure. For example, an acute emergency situation may be detected in the case of the member of remote operating staff in this connection. In addition, providing the control signal or the alternative control signal as a function of the existence of a reduction in suitability (e.g., the emergency situation) of the member of remote operating staff provides the medical device may be safely remotely controlled. A, for example, potentially inexperienced member of on-site operating staff may be supported here (e.g., with the stabilization of the examination object).

In a further embodiment of the method, act a) may include capturing an instantaneous suitability parameter of an alternative medical member of remote operating staff. The alternative member of remote operating staff may be equipped to remotely control the medical device using an alternative remote control unit. In act b), in the case of affirmation, the suitability parameter of the member of remote operating staff and of the alternative member of remote operating staff may be compared. The alternative control signal may be provided as a function of the comparison of the suitability parameter.

The instantaneous suitability parameter of the alternative medical member of remote operating staff may be provided analogously to capture the instantaneous suitability parameter of the member of remote operating staff. In addition, the instantaneous suitability parameter of the alternative member of remote operating staff may have, for example, all features and properties of the instantaneous suitability parameter of the member of remote operating staff. The alternative member of remote operating staff may include a further member of remote operating staff who is equipped to remotely control the medical device using the alternative remote control unit. The alternative remote control unit may have, for example, all features and properties of the remote control unit.

In one embodiment, with the existence of a reduction in the suitability of the member of remote operating staff, the instantaneous suitability parameter of the member of remote operating staff and the alternative member of remote operating staff may be compared in act b). The comparison of the instantaneous suitability parameters may include, for example, forming a difference or a quotient. In one embodiment, the member of remote operating staff or the alternative member of remote operating staff may be identified as the more suitable member of remote operating staff for remotely controlling the medical device based on the comparison of the suitability parameters.

If the comparison reveals that the alternative member of remote operating staff is instantaneously more suitable for remotely controlling the medical device, providing the alternative control signal may include a changeover to remote control of the medical device by the alternative member of remote operating staff using the alternative remote control unit. For example, the medical device may be remotely controlled by the alternative control signal, which is provided by the alternative remote control unit. Alternatively, for example, if the comparison reveals that the member of remote operating staff is instantaneously more suitable for remotely controlling the medical device, the medical device may be provided with the control signal for remotely controlling the medical device.

The embodiment may provide that the medical device may be remotely controlled by the instantaneously most suitable member of remote operating staff, respectively.

In a further embodiment of the method, providing the alternative control signal may inlclude a changeover to remote control of the medical device by an alternative member of remote operating staff using an alternative remote control unit.

With identification of a reduction in the suitability of the member of remote operating staff, the medical device is provided with the alternative control signal in act b). Providing the alternative control signal may include the changeover (e.g., automatic changeover) to remote control of the medical device by the alternative member of remote operating staff using the alternative remote control unit. Providing the alternative control signal may include a switchover (e.g., an automatic routing) from remote control of the medical device by the member of remote operating staff having the reduction in suitability to remote control of the medical device by the alternative member of remote operating staff. In addition, the medical device may be provided with the alternative control signal (e.g., after the changeover or remote control) for remote control of the medical device by the alternative remote control unit.

The changeover to remote control of the medical device by the alternative member of remote operating staff may take place as a function of the comparison of the suitability parameter of the member of remote operating staff and of the alternative member of remote operating staff. In one embodiment, the remote control of the medical device may only be changed over if the alternative member of remote operating staff has a greater suitability compared to the member of remote operating staff.

Alternatively, the changeover to remote control of the medical device by the alternative member of remote operating staff may take place independently of a suitability parameter of the alternative member of remote operating staff.

The embodiment may provide that with the existence of a reduction in the suitability of the member of remote operating staff, the medical device may continue to be remotely controlled by an alternative member of remote operating staff.

In a further embodiment of the method, providing the alternative control signal may include providing a member of on-site operating staff with a workflow indication for the on-site operation of the medical device and/or for carrying out a further procedural step.

In one embodiment, the workflow indication may be provided by a, for example, optical and/or acoustic and/or a haptic output unit (e.g., a display unit and/or a loudspeaker). The workflow indication may have an instruction for on-site operation of the medical device by the member of on-site operating staff (e.g., in text form and/or speech form and/or symbol form). The member of on-site operating staff may include one or more persons (e.g., a female or male doctor) who are arranged in situ (e.g., in close proximity to the examination object and/or in the same room). The workflow indication may include an instruction to assume control of the medical device by the member of on-site operating staff. Alternatively or in addition, the workflow indication may include an instruction for control of the medical device by the member of on-site operating staff.

Alternatively or in addition, providing the alternative control signal may include providing the member of on-site operating staff with a workflow indication to carry out a further procedural step. The further procedural step may include a, for example, dedicated and/or situation-adjusted treatment and/or examination step on the examination object, which is supported and/or executed by the member of on-site operating staff and/or using the medical device. The further procedural step may include administering a medication. Alternatively or in addition, the further procedural step may include arranging and/or moving and/or manipulating a medical object (e.g., a medical instrument, such as a diagnostic and/or surgical instrument) and/or an implant on and/or in the examination object. Alternatively or in addition, the further procedural step may include recording medical image data of the examination object using the medical device (e.g., a medical imaging device). Alternatively or in addition, the further procedural step may include treating at least one treatment area of the examination object (e.g., carrying out an ultrasound, a brachytherapy, shock wave, and/or an irradiation procedure).

The member of on-site operating staff may be informed about the reduction in the suitability of the member of remote operating staff by the workflow indication. In addition, the member of on-site operating staff may be instructed and/or supported by the workflow indication in the on-site operation of the medical device and/or carrying out of the further procedural step (e.g., in the case of a stabilization and/or preparation for transport of the examination object).

In a further embodiment of the method, acts a) and b) may be repeatedly executed until a termination condition occurs.

In one embodiment, the termination condition may specify a maximum number of repetitions of acts a) and b) (e.g., a number or remaining planned procedural steps). Alternatively or in addition, the termination condition may specify a maximum duration of the repeated execution of acts a) and b) (e.g., overall). Alternatively or in addition, identifying the reduction in the suitability of the member of remote operating staff may result in the occurrence of the termination condition (e.g., after the alternative control signal has been provided).

The embodiment may enable repeated monitoring of the remote control suitability.

In a further embodiment of the method, a procedural step may be identified based on procedural planning and/or the control signal. Act b) may include a comparison of a number of attempts at executing the procedural step with a specified maximum value and/or executing the procedural step with a specified desired execution.

In one embodiment, the procedural planning may be received. Receiving the procedural planning may include capturing and/or reading-out a computer-readable data store (e.g., a database). Alternatively or in addition, the procedural planning may be captured based on a user input of a member of medical operating staff (e.g., the member of remote operating staff and/or the member of on-site operating staff). The procedural planning may have an item of information relating to the procedural step (e.g., to a plurality of procedural steps, such as a treatment step and/or examination step), which has been carried out on the examination object (e.g., completely) before the beginning of the method. The procedural step may include administering a medication. Alternatively or in addition, the procedural step may include arranging and/or moving and/or manipulating a medical object (e.g., a medical, such as diagnostic and/or surgical, instrument) and/or an implant on and/or in the examination object. Alternatively or in addition, the procedural step may include recording medical image data of the examination object using the medical device (e.g., a medical imaging device). Alternatively or in addition, the procedural step may include treating at least one treatment area of the examination object (e.g., carrying out an ultrasound, brachytherapy, shock wave, and/or irradiation procedure). In one embodiment, the most recently executed procedural step may be identified based on the procedural planning.

Alternatively or in addition, the procedural step (e.g., the most recently executed procedural step) may be identified using the control signal, which was provided before the beginning of the method for controlling the medical device to execute the procedural step. For example, identifying the procedural step may include a comparison of the control signal with the procedural planning.

In one embodiment, a maximum value may be specified (e.g., received) for a maximum number of attempts at executing the procedural step. Incomplete executions through to complete (e.g., successful) execution of the procedural step for the first time may be counted as an attempt. Further, the number of attempts at executing the procedural step may be counted and be compared with the maximum value. Overshooting of the maximum value by the captured number of attempts at execution of the procedural step may be identified in act b) as a reduction in the suitability of the member of remote operating staff.

In one embodiment, a desired execution for the procedural step may be specified (e.g., received). For example, the procedural planning may include the desired execution for the procedural step (e.g., the plurality of procedural steps). The desired execution may specify at least one procedural parameter of the procedural step. The procedural parameter may include, for example, an administration rate and/or dose and/or composition of the medication. Alternatively or in addition, the procedural parameter may include a movement speed and/or direction of movement and/or positioning (e.g., spatial position and/or orientation and/or pose, and/or trajectory of the medical device). In one embodiment, the execution of the procedural step may be captured (e.g., documented, such as in order to render decisions on the execution of the procedural step comprehensible for a later instant). Further, the captured execution may be compared with the desired execution of the procedural step. A deviation of the desired execution of the procedural step may be identified in act b) as a reduction in the suitability of the member of remote operating staff.

The embodiment may enable procedure-specific monitoring of the remote control suitability.

In a further embodiment of the method, the maximum value and/or the desired execution may be received from a data storage unit. The maximum value and/or the desired execution may be specified in accordance with a standardized or statistical execution of the procedural step.

Receiving the maximum value and/or the desired execution from the data storage unit may include capturing and/or reading-out a computer-readable data store (e.g., a database). According to a first variant, the maximum value and/or the desired execution may be specified in accordance with the standardized execution (e.g., standard operating procedure, SOP) of the procedural step. Alternatively, the maximum value and/or the desired execution may be specified in accordance with a statistical execution of the procedural step. The statistical execution may have been ascertained by analysis of historic and/or simulated executions of the procedural step (e.g., as a statistical mean). The historic executions of the procedural step by the member of remote operating staff or a further member of medical operating staff may have been carried out (e.g., on at least one further examination object).

The embodiment may enable procedure-specific and operator-specific monitoring of the remote control suitability.

In a further embodiment of the method, the maximum value and/or the desired execution may be adapted to a historic operating behavior of the member of remote operating staff for execution of the procedural step.

In one embodiment, the maximum value and/or the desired execution of the procedural step may be received in accordance with a statistical execution of the procedural step, which may have been ascertained, for example, by analysis of historical executions of the procedural step. If the historic executions of the procedural step have been executed (e.g., completely) by the member of remote operating staff, the maximum value and/or the desired execution may be inherently adapted to the historic operating behavior of the member of remote operating staff. If the historic executions of the procedural step have been carried out at least partially (e.g., completely) by a further member of medical operating staff, the maximum value and/or the desired execution may be adapted to the historic operating behavior of the member of remote operating staff. Similarly, the maximum value and/or the desired execution may be adapted to the historic operating behavior of the member of remote operating staff in the case of a specification in accordance with a standard operating procedure of the procedural step.

The historic operating behavior of the member of remote operating staff may include historic executions of the procedural step and/or further procedural steps by the member of remote operating staff (e.g., on the examination object and/or further examination objects). With the historic executions of the procedural step and/or the further procedural steps, one procedural parameter may have been captured respectively. The maximum value and/or the desired execution may be adapted to the historic operating behavior of the member of remote operating staff based on the procedural parameters of the historic executions of the procedural step and/or the further procedural steps (e.g., with the necessary adjustments with respect to the procedural step).

The embodiment may enable procedure-specific and operator-specific monitoring of the remote control suitability.

In a further embodiment of the method, verbal communication of the member of remote operating staff may be captured by the remote control unit and/or the sensor unit. Act b) may include identifying a change in communication. In addition, the existence of a change in communication may be identified as a reduction in the suitability of the member of remote operating staff.

The remote control unit and/or the sensor unit may be configured to capture the verbal communication (e.g., speech and/or text) of the member of remote operating staff. For this, the remote control unit and/or the sensor unit may include an acoustic sensor (e.g., a microphone) and/or an input unit (e.g., a keyboard) that is configured to capture the verbal communication of the member of remote operating staff. The verbal communication may be captured during the course of the capture of a user input and/or in addition (e.g., independently) of the capture of the user input.

In one embodiment, act b) may include identifying a change in communication. For this, the verbal communication of the member of remote operating staff may be repeatedly (e.g., continuously) monitored. For example, an accumulation of speech and/or text errors, a change (e.g., speeding up and/or interruption and/or slowing down) of a flow of speech and/or text and/or an altered choice of words and/or an altered tonality of the member of remote operating staff may be identified as a change in communication. In one embodiment, the existence of the change in communication may be identified as a reduction in the suitability of the member of remote operating staff.

For example, cognitive restrictions on the part of the member of remote operating staff may be identified as a reduction in suitability hereby.

In a further embodiment of the method, the remote control unit may repeatedly query a status of the member of remote operating staff. A delayed, an incorrect, or a missing input of the member of remote operating staff in response to the query may be identified as a reduction in suitability.

In one embodiment, the remote control unit may query a status (e.g., an input) of the member of remote operating staff. For this, the remote control unit may output an acoustic and/or a haptic and/or an optical signal by an output unit (e.g., a loudspeaker and/or vibration device and/or a display unit). The signal may include a request to the member of remote operating staff for a, for example, specific input. The remote control unit may also be configured to capture the input using the input unit. The input may include, for example, a speech input and/or haptic input (e.g., actuation of a button and/or pedal) and/or an input gesture. An absence of the input (e.g., a delayed or missing input) within a specified period following the status query (e.g., following output of the signal) may be identified as a reduction in the suitability of the member of remote operating staff. The status query may require a predetermined input as a desired input. An incorrect input may be identified as a deviation via a comparison between the input of the member of remote operating staff and the desired input. Further, the incorrect input in response to the query may be identified as a reduction in the suitability of the member of remote operating staff.

A responsiveness of the member of remote operating staff that is necessary for the procedure may be monitored hereby.

In a further embodiment of the method, the sensor unit may repeatedly capture a positioning and/or movement of the member of remote operating staff. Act b) may include identifying an atypical positioning and/or movement of the member of remote operating staff. The existence of an atypical positioning and/or movement of the member of remote operating staff may be identified as a reduction in suitability.

In one embodiment, the sensor unit may repeatedly (e.g., continuously) capture the positioning (e.g., spatial position and/or orientation and/or pose) and/or the movement (e.g., a movement speed and/or form of movement and/or direction of movement and/or movement amplitude) of the member of remote operating staff. A change in positioning (e.g., a fall and/or keeling over) and/or a change in the movement (e.g., a slowing down and/or speeding up and/or stopping and/or change in the form of movement and/or direction of movement and/or movement amplitude) may be identified in act b) as an atypical positioning and/or movement of the member of remote operating staff. Identifying the atypical positioning and/or movement of the member of remote operating staff may include a comparison of an instantaneous positioning and/or movement with a previously captured (e.g., historic) positioning and/or movement of the member of remote operating staff. The existence of the atypical positioning and/or movement of the member of remote operating staff may be identified as a reduction in suitability.

For example, physical restrictions on the part of the member of remote operating staff may be identified as a reduction in suitability hereby.

In a further embodiment of the method, the medical device may include a medical imaging device and/or a medical treatment device and/or a remote manipulation unit for robotic movement of a medical object.

The medical imaging device may include, for example, a medical X-ray device and/or a computed tomography system (CT system) and/or a magnetic resonance tomography system (MRT system) and/or an ultrasound device and/or a positron emission tomography system (PET system) that is configured for recording medical image data of the examination object. The medical treatment device may include, for example, an automatic injection machine and/or an irradiation system and/or a brachytherapy device and/or a ventilator and/or a histotripsy device that is configured for treating at least one treatment area of the examination object. The remote manipulation unit (e.g., telemanipulation unit) may include a catheter robot and/or a surgical robot that is configured for robotic movement of the medical object.

The embodiment may enable monitoring of the remote control suitability in the case of remote control of imaging steps, treatment steps, and/or examination steps on the examination object.

In a second aspect, the present embodiments relate to a system including a remote control unit, a provisioning unit, and a medical device. The system is configured to carry out a method for monitoring remote control suitability. The remote control unit is configured to capture a user input of the member of remote operating staff. The provisioning unit is configured to identify, based on the suitability parameter, whether the reduction in the suitability of the member of remote operating staff exists. In the case of affirmation, the provisioning unit provides the medical device with the alternative control signal. In the case of negation, the remote control unit provides the medical device with the control signal as a function of the user input. The medical device is configured to be remotely controlled by the control signal.

The advantages of the system substantially match the advantages of the method for monitoring remote control suitability. Features, advantages, or alternative embodiments mentioned in this connection may likewise also be transferred to the other claimed subject matters, and vice versa.

In a further embodiment of the system, the system may also include a sensor unit that is configured to capture the suitability parameter.

In a further embodiment of the system, the system may also include an alternative remote control unit. The alternative remote control unit may be configured to capture an alternative user input of the alternative member of remote operating staff and, on presentation of a reduction in the suitability of the member of remote operating staff, provide the medical device with the alternative control signal as a function of the alternative user input. The medical device may be configured to be remotely controlled by the alternative control signal.

In a further embodiment of the system, the system may also include an output unit that, on presentation of a reduction in suitability, is configured to provide a member of on-site operating staff with a workflow indication for the on-site operation of the medical device and/or for carrying out a further procedural step.

In a third aspect, the present embodiments relate to a computer program product with a computer program that may be loaded directly into a memory of a provisioning unit, with program segments in order to carry out all acts of the method for monitoring remote control suitability when the program segments are executed by the provisioning unit. The computer program product may include software with a source code that still has to be compiled and linked or only has to be interpreted, or an executable software code that, for execution, only has to be loaded in the provisioning unit. The method for monitoring remote control suitability by a provisioning unit may be carried out quickly, in an identically repeatable manner, and robustly by the computer program product. The computer program product is configured such that the computer program product may carry out the method acts of the present embodiment by the provisioning unit. Apart from the computer program, a computer program product of this kind may optionally include additional constituent parts, such as, for example, documentation and/or additional components, as well as hardware components, such as, for example, hardware keys (e.g., dongles, etc.), in order to utilize the software.

The advantages of the computer program product substantially match the advantages of the method for monitoring remote control suitability. Features, advantages, or alternative embodiments mentioned in this connection may likewise be transferred to the other subject matters, and vice versa.

The present embodiments also include a computer-readable storage medium (e.g., a non-transitory computer-readable storage medium) and/or electronically readable data carrier on which program segments that may be read and executed by a provisioning unit are saved in order to carry out all acts of the method for monitoring remote control suitability when the program segments are executed by the provisioning unit. An implementation largely in terms of software has the advantage that even previously used provisioning units may be easily retrofitted via a software update in order to work according to the present embodiments.

### BRIEF DESCRIPTION OF THE DRAWINGS

Embodiments of the invention are represented in the drawings and will be described in more detail below. Same reference numerals are used in the different figures for same features. In the drawings:

FIGS. 1 to 3 show schematic representations of different embodiments of a method for monitoring remote control suitability; and

FIGS. 4 and 5 show schematic representations of different embodiments of a system.

### DETAILED DESCRIPTION

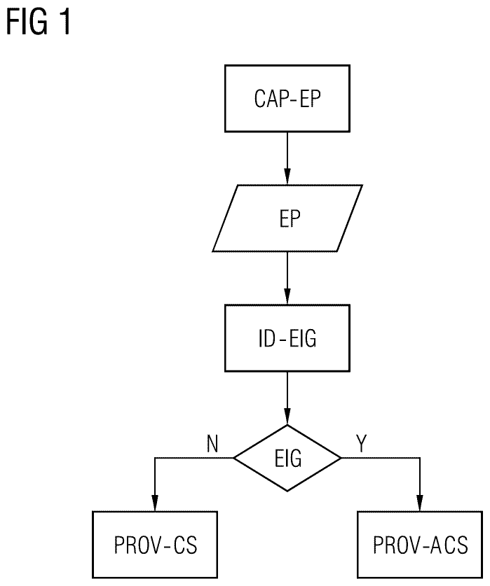

FIG. 1 schematically represents an embodiment of a method for monitoring remote control suitability. In a first act a), an instantaneous suitability parameter EP of a medical member of remote operating staff may be captured CAP-EP. The member of remote operating staff may be equipped to remotely control a medical device using a remote control unit. The medical device may include a medical imaging device and/or a medical treatment device and/or a remote manipulation unit for robotic movement of a medical object. The remote control unit may be configured to provide a control signal to the medical device. The suitability parameter EP may be provided by the remote control unit and/or a sensor unit for capturing the member of remote operating staff. In a second act b), it is possible to identify ID-EIG based on the suitability parameter whether a reduction in the suitability of the member of remote operating staff exists. In case of affirmation Y, the medical device may be provided with an alternative control signal ACS. In case of negation, the medical device may be provided PROV-CS with the control signal CS.

In one embodiment, providing the alternative control signal PROV-ACS may include providing a member of on-site operating staff with a workflow indication for on-site operation of the medical device and/or for carrying out a further procedural step.

In one embodiment, it is possible to identify a procedural step based on a procedural planning and/or the control signal. Act b) may include a comparison of a number of attempts at execution of the procedural step with a specified maximum value and/or an execution of the procedural step with a specified desired execution. The maximum value and/or the desired execution may be received from a data storage unit. The maximum value and/or the desired execution may be specified in accordance with a standardized or statistical execution of the procedural step. In addition, the maximum value and/or the desired execution may be adapted to historical operating behavior of the member of remote operating staff for execution of the procedural step.

In one embodiment, a verbal communication of the member of remote operating staff may be captured by the remote control unit and/or the sensor unit. Act b) may include identifying a change in communication. The existence of a change in communication may be identified as a reduction in the suitability of the member of remote operating staff.

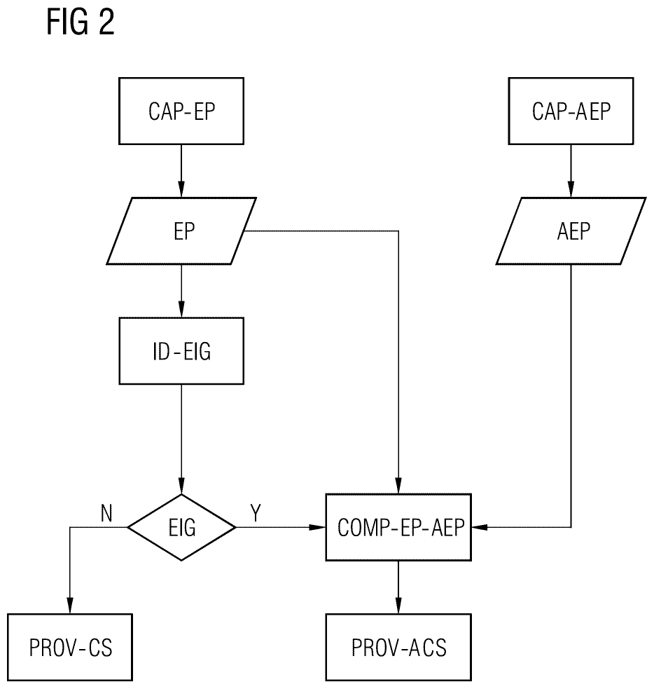

FIG. 2 shows a schematic representation of a further embodiment of the method for monitoring remote control suitability. Act a) may include capturing CAP-AEP an instantaneous suitability parameter AEP of an alternative medical member of remote operating staff. The alternative member of remote operating staff may be equipped to remotely control the medical device using an alternative remote control unit. In act b), in the case of affirmation, the suitability parameter of the member of remote operating staff EP and of the alternative member of remote operating staff AEP may be compared COMP-EP-AEP. In one embodiment, the alternative control signal PROV-ACS may be provided as a function of the comparison of the suitability parameter COMP-EP-AEP.

Providing the alternative control signal PROV-ACS may include a changeover to remote control of the medical device by an alternative member of remote operating staff using an alternative remote control unit.

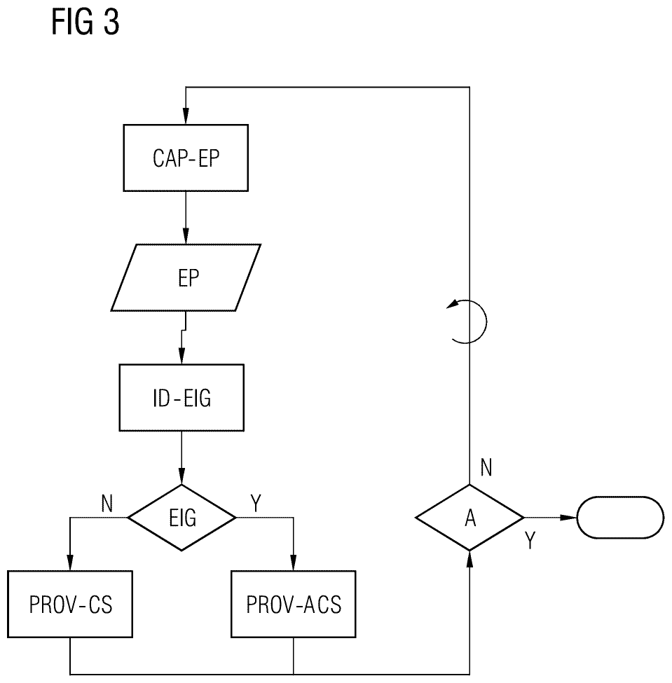

FIG. 3 schematically represents a further embodiment of the method for monitoring remote control suitability. Acts a) and b) may be repeatedly executed until a termination condition A occurs Y.

In one embodiment, the remote control unit may repeatedly query a status of the member of remote operating staff. A delayed, an incorrect, or a missing input of the member of remote operating staff in response to the query may be identified as a reduction in suitability.

The sensor unit may repeatedly capture a positioning and/or movement of the member of remote operating staff. Act b) may include identifying an atypical positioning and/or a movement of the member of remote operating staff. The existence of an atypical positioning and/or movement of the member of remote operating staff may be identified as a reduction in suitability.

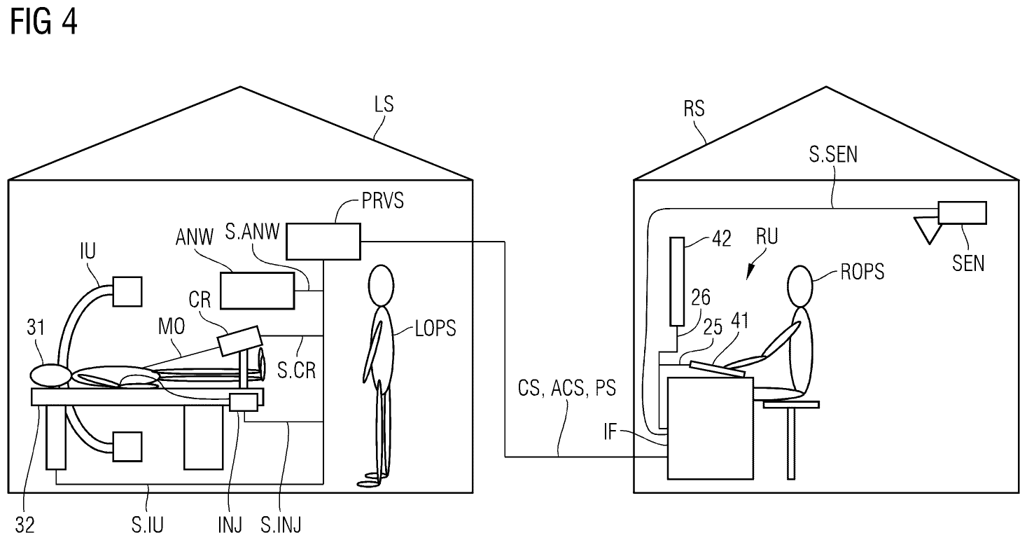

FIG. 4 shows a schematic representation of a proposed system. The system may include a remote control unit RU, a provisioning unit PRVS, and a medical device. The system may be configured to carry out a method for monitoring remote control suitability.

The remote control unit RU may include a user interface (e.g., an input unit 41 and an output unit, such as a display unit 42). The input unit 41 may be configured to capture a user input of a medical member of remote operating staff ROPS. The input unit 41 may include, for example, a keyboard and/or a button and/or a joystick and/or a touchpad and/or a microphone (e.g., for speech capture), and/or a camera (e.g., for capturing gestures). In one embodiment, the input unit 41 may be integrated in the display unit 42 (e.g., in the case of a resistive and/or capacitive input display). The remote control unit RU may, as FIG. 4 illustrates, be arranged spatially and locally spaced apart from the examination object 31 and the medical device. For example, the examination object 31, a member of on-site operating staff LOPS, and the medical device may be arranged in an on-site treatment room LS. Further, the remote control unit RU and the medical member of remote operating staff ROPS may be arranged in a remote control room RS.

For recording medical image data of the examination object 31, a medical imaging device (e.g., a medical C-arm X-ray device IU) may be arranged in the on-site treatment room LS. The C-arm X-ray device IU may be communicatively coupled to the provisioning unit PRVS via a signal S.IU. In one embodiment, the C-arm X-ray device IU (e.g., recording of the image data and/or a movement of the C-arm X-ray device IU) may be remotely controlled by a control signal CS from the remote control unit RU (e.g., as a function of a user input of the medical operating staff OPS). The provisioning unit PRVS may receive the medical image data based on the signal S.IU, and provide the remote control unit RU with the medical image data using a provision signal PS. The display unit 42 may be configured to output a graphical representation of the medical image data. For this, the interface IF may receive the provision signal PS and send a corresponding signal 26 to the display unit 42.

In one embodiment, the system may also include a sensor unit SEN that is configured to capture the suitability parameter EP. The sensor unit SEN may include, for example, an optical sensor (e.g., a camera) and/or an acoustic sensor (e.g., a microphone) and/or a mechanical sensor (e.g., a push button) that is configured to capture the member of remote operating staff (e.g., a positioning and/or change in positioning and/or of a bodily and/or cognitive state of the member of remote operating staff ROPS). In addition, the sensor unit may provide the instantaneous suitability parameter EP as a function of the captured positioning and/or change in positioning and/or of the captured bodily and/or cognitive state of the member of remote operating staff ROPS, for example, using a signal S.SEN.

The provisioning unit PRVS may be configured to identify ID-EIG, based on, the suitability parameter EP whether the reduction in the suitability of the member of remote operating staff ROPS exists. With the existence of a reduction in suitability, the provisioning unit PRVS may provide the medical device with the alternative control signal ACS. With non-existence of a reduction in suitability, the remote control unit RU may provide the medical device with the control signal CS as a function of the user input. The medical device may be configured to be remotely controlled by the control signal.

In one embodiment, the medical device may include a movement apparatus CR (e.g., a catheter robot) and an automatic injection machine INJ. The movement apparatus CR may be communicatively coupled to the provisioning unit PRVS by a signal S.CR and the automatic injection machine by a signal S.INJ. The movement apparatus CR may be configured for robotic movement (e.g., for translation and/or rotation) of a medical object MO (e.g., a catheter and/or surgical instrument) that, in an operating state of the system, is at least partially arranged in the examination object 31. Further, the remote control unit RU may be configured (e.g., by the control signal CS) to control the robotic movement of the medical object MO as a function of the user input. For example, the remote control unit RU may be configured to release or adjust or stop the robotic movement of the medical object MO (e.g., as a function of the user input).

The automatic injection machine INJ may be configured to administer a medication (e.g., a contrast agent). The remote control unit RU may be configured (e.g., using the control signal) to adjust a rate of administration and/or dose and/or composition of the medication and/or release or stop the administration of the medication as a function of the user input.

In one embodiment, the system may also include an output unit ANW that, on presentation of a reduction in suitability, is configured to provide a workflow indication for the on-site operation of the medical device (e.g., the movement apparatus CR and/or of the automatic injection machine INJ) and/or for carrying out a further procedural step to the member of on-site operating staff LOPS. The output unit ANW may include, for example, a loudspeaker and/or a further display unit. Further, the output unit may be communicatively coupled to the provisioning unit PRVS by a signal S.ANW.

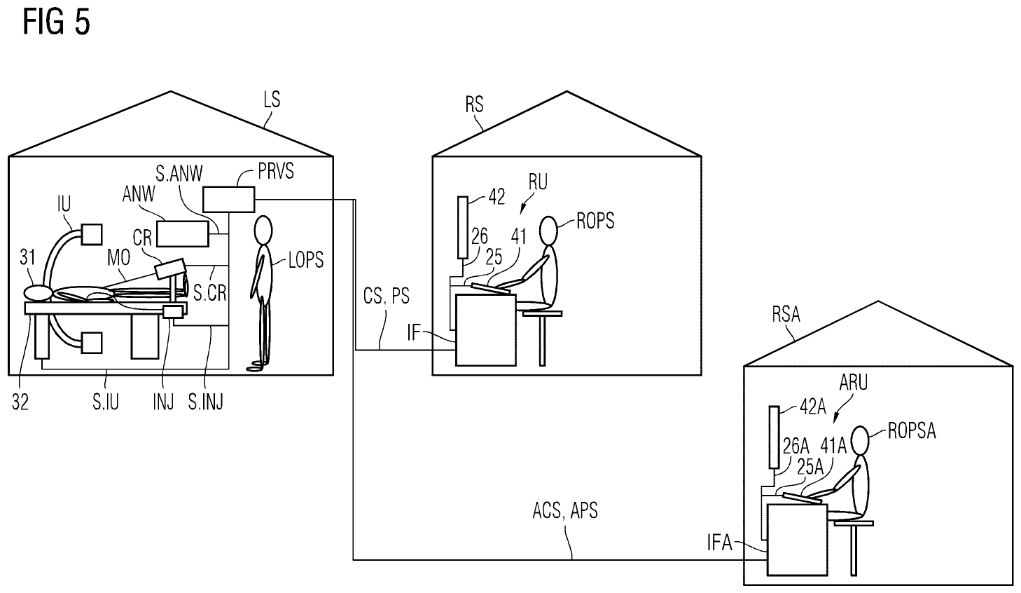

FIG. 5 shows a schematic representation of a further embodiment of a proposed system. The system may also include an alternative remote control unit ARU. The alternative remote control unit ARU may be configured to capture an alternative user input of an alternative member of remote operating staff ROPSA.

The alternative remote control unit ARU may include a further user interface IFA (e.g., a further input unit 41A) and a further output unit (e.g., a further display unit 42A). The further input unit 41A may be configured to capture the further user input of the alternative medical member of remote operating staff ROPSA. The further input unit 41A may include, for example, a keyboard and/or a button and/or a joystick and/or a touchpad and/or a microphone (e.g., for speech capture) and/or a camera (e.g., for capturing gestures). In one embodiment, the further input unit 41A may be integrated in the further display unit 42A, for example, in the case of a resistive and/or capacitive input display. The further remote control unit ARU may, as FIG. 5 illustrates, be arranged spatially and locally spaced apart from the examination object 31 and the medical device. The further remote control unit ARU and the further member of remote operating staff ROPSA may be arranged in a further remote operating room RSA.

The provisioning unit PRVS may receive the medical image data based on the signal S.IU and provide the alternative remote control unit ARU with the medical image data using a further provision signal APS. The further display unit 42A may be configured to output a graphical representation of the medical image data. For this, the further interface IFA may receive the further provision signal APS and send a corresponding signal 26A to the further display unit 42A.

The schematic representations contained in the described figures do not depict any kind of scale or size ratios.

In the context of the present application, the expression “on the basis of” may be understood, for example, within the meaning of the expression “using”. For example, wording, according to which a first feature is generated (e.g., alternatively, ascertained, determined, etc.) based on a second feature, does not preclude the first feature from being generated (e.g., alternatively, ascertained, determined, etc.) based on a third feature.

The preceding methods described in detail and the represented apparatuses are merely embodiments that may be modified in a variety of ways by a person skilled in the art without departing from the scope of the invention. Further, use of the indefinite article “a” or “an” does not preclude the relevant features from also being present multiple times. Similarly, the terms “unit” and “element” do not preclude the relevant components from being composed of a plurality of cooperating sub-components, which may optionally also be spatially distributed.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for monitoring remote control suitability, the method comprising:
capturing an instantaneous suitability parameter of a medical member of remote operating staff, wherein the member of remote operating staff is equipped to remotely control a medical device using a remote control unit, wherein the remote control unit is configured to provide the medical device with a control signal, and wherein the instantaneous suitability parameter is provided by the remote control unit, a sensor unit for capturing the member of remote operating staff, or the remote control unit and the sensor unit;
identifying, based on the instantaneous suitability parameter, whether a reduction in the suitability of the member of remote operating staff exists;
in the case of affirmation in the identifying, providing the medical device with an alternative control signal; and
in the case of negation in the identifying, providing the medical device with the control signal.

2. The method of claim 1, wherein the capturing comprises capturing an instantaneous suitability parameter of an alternative medical member of remote operating staff,
wherein the alternative member of remote operating staff is equipped to remotely control the medical device using an alternative remote control unit,
wherein in the case of affirmation in the identifying, the method further comprises comparing the instantaneous suitability parameter of the member of remote operating staff and the instantaneous suitability parameter of the alternative member of remote operating staff, and
wherein the instantaneous alternative control signal is provided as a function of the comparison of the suitability parameters.

3. The method of claim 1, wherein providing the alternative control signal comprises a changeover to remote control of the medical device by an alternative member of remote operating staff using an alternative remote control unit.

4. The method of claim 1, wherein providing the alternative control signal comprises providing a member of on-site operating staff with a workflow indication for on-site operation of the medical device, for carrying out a further procedural step, or for a combination thereof.

5. The method of claim 1, wherein the capturing and the identifying are repeatedly executed until a termination condition occurs.

6. The method of claim 1, further comprising identifying a procedural step using procedural planning, the control signal, or the procedural planning and the control signal,
wherein the identifying comprises comparing a number of attempts to execute the procedural step with a specified maximum value, execution of the procedural step with a specified desired execution, or a combination thereof.

7. The method of claim 6, further comprising receiving the maximum value, the desired execution, or the combination thereof from a data storage unit,
wherein the maximum value, the desired execution, or the combination thereof is specified according to a standardized or statistical execution of the procedural step.

8. The method of claim 6, further comprising adapting the maximum value, the desired execution, or the combination thereof to a historic operating behavior of the member of remote operating staff for execution of the procedural step.

9. The method of claim 1, wherein a verbal communication of the member of remote operating staff is captured by the remote control unit, the sensor unit, or the remote control unit and the sensor unit,
wherein the identifying comprises identifying a change in communication, and
wherein the existence of a change in communication is identified as a reduction in a suitability of the member of remote operating staff.

10. The method of claim 1, wherein the remote control unit repeatedly queries a status of the member of remote operating staff, and
wherein a delayed, an incorrect, or a missing input by the member of remote operating staff in response to the query is identified as a reduction in suitability.

11. The method of claim 1, wherein the sensor unit repeatedly captures a positioning, movement, or positioning and movement of the member of remote operating staff,
wherein the identifying comprises identifying an atypical positioning, movement, or positioning and movement of the member of remote operating staff, and
wherein the existence of an atypical positioning, a movement, or atypical positioning and movement of the member of remote operating staff is identified as a reduction in suitability.

12. The method of claim 1, wherein the medical device comprises a medical imaging device, a medical treatment device, a remote manipulation unit for robotic movement of a medical object, or any combination thereof.

13. A system comprising:
a remote control unit;
a provisioning unit; and
a medical device configured to monitor remote control suitability, the medical device being configured to monitor the remote control suitability comprising the medical device being configured to:
capture an instantaneous suitability parameter of a medical member of remote operating staff, wherein the member of remote operating staff is equipped to remotely control the medical device using the remote control unit, wherein the remote control unit is configured to provide the medical device with a control signal, and wherein the instantaneous suitability parameter is provided by the remote control unit, a sensor unit for capturing the member of remote operating staff, or the remote control unit and the sensor unit,
wherein the remote control unit is configured to capture a user input of the member of remote operating staff,
wherein the provisioning unit is configured to identify, based on the instantaneous suitability parameter, whether reduction in the suitability of the member of remote operating staff exists,
wherein in the case of affirmation in the identification, the provisioning unit is configured to provide the medical device with the alternative control signal,
wherein in the case of negation in the identification, the remote control unit is configured to provide the medical device with the control signal as a function of the user input, and
wherein the medical device is configured to be remotely controlled by the control signal.

14. The system of claim 13, further comprising the sensor unit configured to capture the instantaneous suitability parameter.

15. The system of claim 13, further comprising:
an alternative remote control unit,
wherein the alternative remote control unit is configured to:
capture an alternative user input of an alternative member of remote operating staff; and
on presentation of a reduction in the instantaneous suitability of the member of remote operating staff, provide the medical device with the alternative control signal as a function of the alternative user input,
wherein the medical device is further configured to be remotely controlled by the alternative control signal.

16. The system of claim 13, further comprising an output unit that, on presentation of a reduction in the instantaneous suitability of the remote operating staff, is configured to provide a member of on-site operating staff with a workflow indication for on-site operation of the medical device, for carrying out a further procedural step, or for a combination thereof.

17. In a non-transitory computer-readable storage medium that stores instructions executable by one or more processor to monitor remote control suitability, the instructions comprising:
capturing an instantaneous suitability parameter of a medical member of remote operating staff, wherein the member of remote operating staff is equipped to remotely control a medical device using a remote control unit, wherein the remote control unit is configured to provide the medical device with a control signal, and wherein the instantaneous suitability parameter is provided by the remote control unit, a sensor unit for capturing the member of remote operating staff, or the remote control unit and the sensor unit;
identifying, based on the instantaneous suitability parameter, whether a reduction in the suitability of the member of remote operating staff exists;
in the case of affirmation in the identifying, providing the medical device with an alternative control signal; and
in the case of negation in the identifying, providing the medical device with the control signal.
